(19) United States
(12) Plant Patent Application Publication
(10) Pub. No.: US 2006/0041978 P1
(43) Pub. Date: Feb. 23, 2006

(54) Double impatiens plant named 'Cameo Pink Chiffon'

(75) Inventor: Xenia Cascante, Alajuela (CR)

Correspondence Address: THE WEBB LAW FIRM, P.C., 700 KOPPERS BUILDING, 436 SEVENTH AVENUE, PITTSBURGH, PA, 15219, US

(73) Assignee: Oglevee, Ltd., Connellsville, PA (US)

(21) Appl. No.: 11/209,365

(22) Filed: Aug. 23, 2005

Related U.S. Application Data
(60) Provisional application No. 60/603,601, filed on Aug. 23, 2004.

(51) Int. Cl. A01H 5/00

(52) U.S. Cl. PLT/317

(57) ABSTRACT

A new and distinct cultivar of Double Impatiens plant named ‘Cameo Pink Chiffon’, characterized by its compact, mounded and outwardly spreading plant habit; rapid growth rate; freely branching habit; dense foliage; freely flowering habit; fully double flower form; and large pink flowers that are positioned above and beyond the foliage.

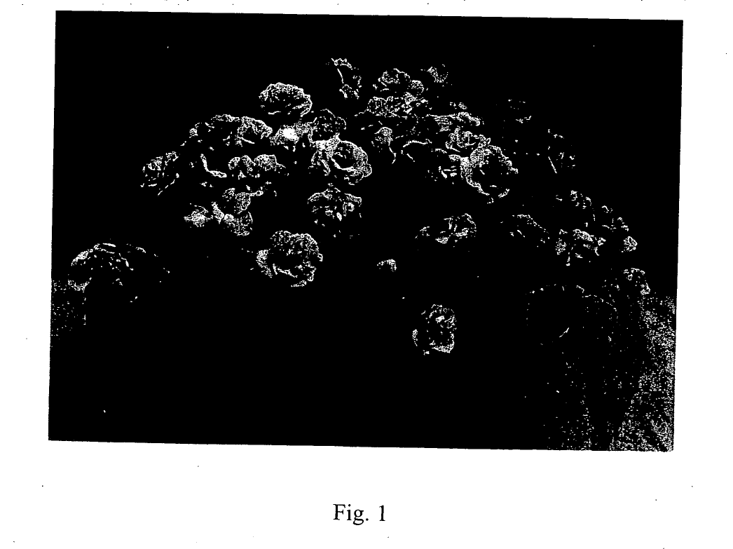

### CROSS-REFERENCE TO RELATED APPLICATIONS

This application claims the benefit of U.S. Provisional Plant Patent Application No. 60/603,601 filed Aug. 23, 2004, which is hereby incorporated by reference in its entirety.

### BOTANICAL CLASSIFICATION

Impatiens walleriana

### VARIETAL DENOMINATON

‘Cameo Pink Chiffon’

### BACKGROUND OF INVENTION

The present invention relates to a new and distinct cultivar of Impatiens walleriana, and hereinafter referred to by the varietal name ‘Cameo Pink Chiffon’. The new variety is a product of a planned breeding program conducted in Cartago, Costa Rica. The purpose of the breeding program was to develop compact and mounded cultivars with fully double flowers, large flower size, and interesting flower colors. The new variety originated from a cross made by the Inventor of two proprietary selections of Impatiens walleriana: IDXCCH-1 (female parent) (unpatented) and IDXCCH-4 (male parent) (unpatented). Comparisons between the new variety and its parents cannot be made, as the parental varieties no longer exist. The cultivar ‘Cameo Pink Chiffon’ was discovered and selected as a flowering plant within the progeny of the stated cross in a controlled environment in Cartago, Costa Rica. Asexual reproduction of the new variety by terminal cuttings has shown that the unique features of the new variety are stable and reproduced true to type in successive propagations.

The following traits have been repeatedly observed and are determined to be the unique characteristics of ‘Cameo Pink Chiffon’. Characteristics that distinguish ‘Cameo Pink Chiffon’ as a new and distinct cultivar include:

1) Large flower size of about 5.0 cm. in diameter;

2) Compact, mounded, spreading growth habit;

3) Freely branching habit;

4) Rounded leaves with dense foliage;

5) Time to produce a finished plant in a 10 cm. pot is 5 to 6 weeks; and

6) Pink flowers are carried above the leaf canopy and face upward.

Plants of the new variety can be compared to plants of the Impatiens walleriana cultivar ‘Cameo Light Pink’, disclosed in U.S. Plant Pat. No. 12,037. In side-by-side comparisons conducted in Lompoc, Calif., plants of the new variety differed from plants of the cultivar ‘Cameo Light Pink’ in the following characteristics:

1) Plants of the new variety had more petals per flower than plants of ‘Cameo Light Pink’;

2) Plants of the new variety had leaves that were longer and wider than plants of ‘Cameo Light Pink’;

3) Plants of the new variety had a stem color of 146D with a cast of 184D on the older stems, whereas plants of the cultivar ‘Cameo Light Pink’ had a stem color of 146C with a heavier cast of 184D;

4) Plants of the new variety had flowers with a color of 69A with a color of 67A at the base of the petals, whereas plants of ‘Cameo Light Pink’ had flowers with a color of 65A; and

5) Plants of the new variety had a flower bud diameter of about 15 mm., whereas plants of ‘Cameo Light Pink’ had a flower bud diameter of about 12 mm.

### BRIEF DESCRIPTION OF THE DRAWING

The accompanying colored photograph illustrates the new variety, with the colors being as nearly true as is possible to obtain in colored illustrations of this type.

### DETAILED BOTANICAL DESCRIPTION

The cultivar ‘Cameo Pink Chiffon’ has not been observed under all possible environmental conditions. The phenotype may vary somewhat with variations in environment such as temperature, day length and light intensity. However, there is no variance in genotype.

In the following description, color references are made to The Royal Horticultural Society Colour Chart, 1995 Edition, except where general terms of ordinary dictionary significance are used. Plants were grown under commercial practice during the late winter through early spring of 2003 in Lompoc, Calif., in a corrugated polycarbonate-covered greenhouse with 30% shade covering. During the production of the plants, day temperatures ranged from 21 to 24° C.; night temperatures ranged from 16 to 18° C.; and light levels ranged from 4000 to 5000 foot candles of light. Plants used in the following description were grown in 15 cm containers for about nine to ten weeks after planting rooted cuttings.
Propagation:
   Type cutting.—Terminal cuttings.
   pi Time to initiate roots.—Summer: About 10 days at 21° C. Winter: About 12 days at 21° C.
   Time to produce a rooted cutting.—Summer: About 21 days at 21° C. Winter: About 21 days at 21° C.
   Root description.—Fibrous, fine, white in color.
   Root habit.—Freely branching, dense.
Plant description:
   General appearance.—Compact, mounded and outwardly spreading. Appropriate for 10-15 cm. pots and 20 to 25 cm. hanging basket containers.
   Growth and branching habit.—Vigorous and freely-branching habit with at least 10 lateral branches developing per plant, dense growth. Pinching (removal of terminal apex) is usually not required.
   Crop time.—Relatively rapid growth rate. From planting one rooted cutting in a 10 cm. pot: About 5 to 6 weeks. From planting three rooted cuttings in a 25 cm. hanging basket container: About 8 to 10 weeks.
   Plant height at flowering.—About 18 to 20 cm.
   Lateral branch description.—Length: About 20 to 25 cm. Diameter: About 8 to 10 mm. Internode length: About 1 to 4 cm. Texture: Smooth, glabrous. Color: 146D with a cast of 184C on older stems.
   Foliage description.—Arrangement: Alternate, simple; dense foliage. Length: About 5 to 6 cm. Width: About 3 to 3.5 cm. Shape: Ovate. Apex: Acute. Base: Acute. Margin: Slightly serrated with fine ciliation. Texture, upper and lower surfaces: Glabrous. Venation pattern: Pinnate. Color: Developing leaves, upper surface: 146B. Developing leaves, lower surface: 146D. Fully expanded leaves, upper surface: 146A. Fully expanded leaves, lower surface: 147C. Venation, upper surface: 146B. Venation, lower surface: 147B. Petiole length: About 12 to 15 mm. Petiole diameter: About 3 mm. Petiole texture upper and lower surface: Smooth, glabrous. Petiole color: 148B.
Flower description:
   Flower type and habit.—Numerous and consistently double pink flowers. Freely and continuously flowering. Flower buds open similar to a rose in fullness; flowers rounded. Usually 8 to 15 flowers and flower buds per lateral branch. Flowers positioned above the foliage and typically face outward to upward. Flowers last about 7 to 10 days on the plant. Flowers not persistent. Flowers not fragrant.
   Flowering season.—Year-round under greenhouse conditions. In the garden, flowering from spring until fall. Flowering indeterminate and continuous.
   Flower diameter.—About 5 cm.
   Flower depth (height).—About 15 to 18 mm.
   Flower buds.—Rate of opening: From full color bud to full open flower, about 5 to 7 days is typical depending on temperature. Length: About 18 mm. Diameter: About 15 mm. Shape: Obovate Color (just before opening): 75C.
   Petals.—Quantity/arrangement: About 30 to 35; imbricate. Length, outer petals: About 25 mm. Width, outer petals: About 22 mm. Shape: Rounded to cordate. Apex: Rounded with indentations. Base: Acute to rounded. Margin: Entire. Texture, upper and lower surfaces: Glabrous, smooth. Color: When opening, upper surface: 68B with 67A at the base. When opening, lower surface: 73D with 67A at the base. Fully opened, upper surface: 69A fading to 69B with 67A at the base. Fully opened, lower surface: 69C with 67A at the base.
   Peduncles.—Length: About 30 to 35 mm. Diameter: About 2 mm. Angle: About 45° to the lateral branch. Strength: Strong. Texture: Smooth, glabrous. Color: 146D.
Reproductive organs: None observed.
Fruit/Seed development: Fruit and seed development has not been observed.
Disease/Pest resistance: Plants of the new variety have not been observed to date to be resistant to pathogens and pests common to Impatiens.
Temperature tolerance: Plants of the new variety have been observed to be tolerant to rain, wind, and temperatures ranging from 5 to 38° C.

I claim:

1. A new and distinct variety of Impatiens named ‘Cameo Pink Chiffon’ as herein described and illustrated.
